(19) United States
(12) Patent Application Publication
(10) Pub. No.: US 2007/0077305 A1
(43) Pub. Date: Apr. 5, 2007

(54) Biocompatible polymeric matrix and preparation thereof

(76) Inventors: Tien Canh Le, Montreal (CA); Tu Hao Tran, Laval (CA); Mircea-Alexandru Mateescu, Montreal (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/240,578

(22) Filed: Oct. 3, 2005

(51) Int. Cl. A61K 9/14

(52) U.S. Cl. 424/488

(57) ABSTRACT

The invention discloses a biocompatible polymeric matrix which is functionalized through the reaction with a functionalizing agent including protonated carboxylic acid groups. There is also disclosed a method of preparation of the polymeric matrix and a pharmaceutical composition including the matrix as a carrier for controlled release of a bioactive agent. The pharmaceutical composition is suitable for immobilizing and protecting the bioactive agent from denaturing factors, and can take various forms such as tablets, spheres, films, hydrogels, and emulsions.

### BACKGROUND OF THE INVENTION

1. Technical Field

The present invention discloses a type of biocompatible polymeric matrix which is functionalized to include carboxylic acid groups, a method of preparation of the polymeric matrix and a composition including the matrix as a carrier of a bioactive agent in various forms such as tablets, spheres, films, hydrogel, emulsions, etc.

1. Description of the Prior Art

There is a great need for polymer matrices able to protect and deliver orally administered bioactive agents, particularly small molecules such as peptides, antigens, drugs, etc. The main difficulties with the oral administered biopharmaceuticals include the fact that many bioactive agents are unstable in the gastrointestinal tract, particularly this results from various denaturant factors, including gastric acidity, proteolytic enzymes, bile acids or compounds present in certain food. The gastric acidity can inactivate certain bioactive agents particularly bioactive peptides and proteins during stomachal transit. Furthermore, biopharmaceuticals tend to be sensitive to the oxygen species and have a short half-life. Thus some biopharmaceuticals tend to diffuse poorly through to the intestinal tissue and thus are not delivered where they are needed.

The oral bioavailability of most peptides and proteins is less than 1%. The reasons for this are poor absorption of the peptides and proteins in the gastrointestinal tract and their degradation by proteolytic enzymes (pepsin, trypsin, chymotrypsin, etc). Furthermore, the absorption of proteins and peptides is different, at different regions of the intestine. The morphology of the intestine changes from one region to another and the proteolytic activity of proteases gradually decrease from the duodenum to the large intestine. This suggests that there may be an optimal site for peptides and proteins release in the small intestine and that the selective delivery into the small intestine is necessary.

The use of polymers as matrices to protect the active ingredients has been considered. Synthetic polymeric materials such as azopolymers (Ghandehari, H. et al., 1997. Biomaterials, 18, 861-872), poly(alkyl cyanoacrylates) (Gao, H. et al., 2004. World J. Gastroenterol., 10, 2010-2013) and graft copolymers with hydrophobic and hydrophilic branches (Sakuma, S. et al., 1997. Int. J. Pharm. 149, pp. 93-106, 1997) or hydrophilic backbone and hydrophobic branches (Le Tien, C. et al., 2003. J. Control. Release, 93, 1-13) have been used to fabricate delivery systems with specific functions.

Copolymer networks were also considered such as polymethacrylic acid (PMA) grafted to polyethylene glycol (PEG), which are hydrogels exhibiting hydrogen bonding designed to achieve such specific functions in oral delivery of peptides and proteins (Klier, J. et al. 1990. Macromolecules, 23, 4944-4949; Lowman, A. M. and Peppas, N. A., 1997. Macromolecules, 30, 4959-4965). These hydrogels were shown to exhibit particular swelling behavior due to the formation of complexes in acidic media via hydrogen bonding between etheric groups of the PEG chains and the protons of the carboxylic groups on the PMA network. Such polymers have been studied as the central core of a drug delivery system in which the polymer-insulin matrix is surrounded by a membrane containing grafted glucose oxidase, which provides the reaction conditions for a change in pH necessary to enhance biodegradation and subsequent insulin delivery (Brannon-Peppas, L. 1997. Med. Plast. Biomater., 4, 34-44).

Natural polymeric materials have equally been considered for improving the stability of molecules during their gastro-intestinal passage. These natural polymers as chitosan, alginate or agarose, etc. present several advantages. For instance, they are non-toxic, biocompatible and easy to obtain in various forms such as tablets, beads or microbeads, granules, etc. Tablet form is preferred by pharmaceutical industries due to their efficient and simple method of production. They can be prepared by mixing a dry powder of the bioactive agents with the matrix, then compressing the dry mixture of powders into a mould or die machine under suitable pressure.

There is increasing interest in the natural material, alginate as a matrix for bioactive agents due to its biocompatibility and low toxicity. A further important feature of alginate is its ionotropic-gelation (Smidsrod, O. and Skjak-Bræk, G. 1990. TIBTECH., 8, 71-78), induced by divalent (i.e. Ca²⁺) or multivalent cations, which ionically cross-link carboxylate groups in the uronate blocks of alginate to produce a gel insoluble at low pH, but becoming soluble at a neutral pH or higher. This behavior affords interesting advantages to use alginate as support for bacteria entrapment, which prevents the solubilization of beads in stomach and gives moderate protection of cells against acid shock. In addition, its great solubility at intestinal pH allows the release of viable cells into the intestinal tract.

A chitosan-alginate network structure has also been reported (Vandenberg, G. W. et al. 2001. J. Control. Release, 77, 297-307; El-Kamel, A. et al., 2002. AAPS Pharm. Sci., 4, 1-7), which constitutes a way of reducing the diffusion phenomenon and, consequently, limits the access of gastric acid to beads.

Chitosan, a poly(2-amino-2-deoxy-β-D-glucopyranose) was reported to exhibit protective effects on the viability of certain cell types (Groboillot, A. F. et al. Biotechnology and Bioengineering, V.42 pp. 1157-1163, 1993) and potential applications for drug delivery (Block and Sabnis, U.S. Pat. No. 5,900,408). Furthermore, the bioadhesive properties of chitosan could enhance transmucosal absorption of peptides or proteins via interactions of positive charges of chitosan with negative charges of sialic acid residues of the mucin present in mucus. When administered to mucosal membranes, chitosan has been demonstrated to be bioadhesive, non-toxic and biocompatible (Hirano, S. et al., 1991, Cosmetic and Pharmaceutical Applications of Polymers, Eds. Gebelein et al, Plenum Press, pp. 95-104)

There is also interest in modifying the chitosan, particularly with regard to its free amino groups in order to improve its solubility under certain specific circumstances.

Nordquist et al. (U.S. Pat. No. 5,747,475) described the chitosan modification by addition of a monosaccharide or an oligosaccharide (N-glycation) to its free amino groups, and its use as an immunoadjuvant (U.S. Pat. No. 5,633,025/1997) proposed the use of carboxymethylchitosan or glycolchitosan as a coating agent.

Aiba (JP 62288602 A2/1987) describes the production of modified chitosan nanoparticles useful as a capturing agent of metal ions, enzyme immobilizing or drug sustained release carriers, etc. These nanoparticles are obtained by atomization of chitosan solution in an alkaline medium and then, by treatment of these nanoparticles in functionalizing solutions such as phosphorus oxichloride, acetaldehyde, glutaraldehyde, etc.

Le-Tien et al. (WO02094224 A1) reported that the chitosan derivatized by N-acylation with fatty acids presents a hydrophobic character, thus improving the resistance of the polymer to the gastric acidity, and allowing it be used for protection and controlled release of sensitive bioactive agents. The acylated acyl chitosan was studied by K. Y. Lee et al. (1995, Biomaterials 16, pp. 1211-1216) chitosan was treated with acylating reagents such as carboxylic acids anhydride (i.e. acetic, propionic, n-butyric, n-valeric or n-hexanoic anhydrides). Chitosan was found to be biodegradable and biocompatable. Several researchers studied the structure of acylated polymers (Desbrieres, J. et al., 1996, Int. J. of Macromolecules, V. 19, pp. 21-28) and showed their structure remained in hydrophobic self-assembling.

The drug dissolution rate of controlled release in matrix systems is frequently governed by diffusion, swelling and/or an erosion mechanism (Brannon-Peppas, L. 1997. Med. Plast. Biomater., 4, 34-44). The rate of diffusion is based on the solvent access inside the matrix, followed by the active ingredient solubilization, and its diffusion through the polymeric structure. The rate of swelling involves several different processes. When in contact with the dissolution medium, the polymer is quickly hydrated and generates a gelled barrier (hydrogel) that gradually advances. This hydration involves significant matrix swelling, enabling the bioactive molecules to diffuse through this barrier. The erosion mechanism rate is limited by bulk dissolution and/or hydrolysis where the polymer degrades in a fairly uniform manner through the matrix and at the same time, the bioactive agent is released in the medium.

The oral route is considered to be the most convenient for drug administration in therapy of chronic diseases, avoiding pain, stress and the risk (infections, hematoma) of daily injections and leading to a better patient compliance.

In the last decade, several reports mentioned the possibilities of oral administration of peptides. The main approaches for peptides oral administration (Gowthamarajan, K. and Kulkarni, G. T., 2003. Resonance, 8, 38-46) were:

Protecting bioactive peptides from enzymatic degradation by using antiproteolytic agents (protease inhibitors) associated with orally administered therapeutic peptides and proteins in order to reduce their proteolytic breakdown by enzymes in the gastrointestinal tract. However, formulations of bioactive peptides (i.e. insulin) with protease inhibitors (i.e. aprotinin) showed inconsistent effects, with different in vitro and in vivo effects;

Promoting gastrointestinal absorption of bioactive peptides through simultaneous use of penetration enhancers in order to increase the absorption of peptides and proteins in the gastrointestinal tract by their action on transcellular and paracellular pathways. Penetration enhancers include surfactants, fatty acids, bile salts and citrates salts, as well as chelators like ethylene diamine tetraacetate (EDTA). Surfactants and fatty acids affect the transcellular pathway by altering membrane lipid organization and increasing thus the absorption of peptides consumed orally. Bile salt micelles, EDTA and trisodium citrate as well as cyclodextrin have been reported to increase the absorption of insulin. A significant increase in the bioavailability of insulin can be achieved by the co-administration of protease inhibitors and penetration enhancers. The limitation with penetration enhancers is lack of specificity, which may lead to long-term toxic effects. Surfactants can cause lysis of mucous membrane and may thus damage the lining of the gastrointestinal tract. Similarly, chelators such as EDTA cause depletion of Ca²⁺ ions, which may in turn cause disruption of actin filaments and thus damage the cell membrane;

Chemical modification of bioactive peptides in order to improve their stability against enzymatic degradation and to enhance their bioavailability. However, chemical modification does not always lead to improved oral absorption. For example, diacyl derivatives of insulin exhibited a higher proteolysis than native insulin in the small intestine of the rat. Moreover, this approach is less applicable due to the inactivation of the biological activity;

Bioadhesive delivery systems for enhancement of contact of the drug with the mucous membrane lining the gastrointestinal tract. The anchoring of a drug formulation to the wall of the gastrointestinal tract increases the overall time available for drug absorption. Bioadhesive polymers such as polycarbophil and chitosan have been reported to improve the oral absorption of peptides; and

Carrier systems such as microspheres, nanoparticles and liposomes can improve the bioavailability of peptides and the oral absorption of peptides and proteins. The introduction of liposomes as a drug delivery system in the late 1980's renewed interest in the oral administration of insulin in the upper gastrointestinal tract and enhancing its absorption from various regions of the small intestine.

Some drugs cannot be given orally because they have no absorption via the intestinal walls. They can, however, be encapsulated in nanoparticles for parenteral administration or entrapped in films for transdermal applications. These forms are of interest for release of steroids, antibiotics, analgesics, etc.

The food formulations as packing or coating films as well as beads or microbeads forms (e.g. bacteriocine entrapment in the microbeads) can protect food products (e.g. chopped meat) against any contamination from pathogenic bacteria.

In this context, there is a need for new carriers and protective polymeric matrices, that are biocompatible, acceptable to regulatory authorities and consumers, preferably biodegradable and compatible for use in pharmaceutics, nutraceutics, cosmetics, as well as in the agriculture and food industry.

### SUMMARY OF THE INVENTION

In one aspect of the invention there is provided a biocompatible polymeric matrix for immobilizing a bioactive agent comprising: a biocompatible polymer comprising at least one polymer chain having a plurality of chain lengths and subunits in the chain lengths, each subunit having at least one reactive functionality and a plurality of pairs of first and second linkages, each pair of linkages extending between a pair of subunits on opposed spaced apart chain lengths, the first and the second linkages having a binding group covalently attached to the reactive functionality and at least one —COOH moiety, distal the binding group, the at least one —COOH moiety of a pair of first and second linkages being bondingly associated such that the chain lengths and the pairs of linkages define the matrix.

In another aspect of the invention there is provided a method for preparing a biocompatible polymeric matrix comprising: a biocompatible polymer comprising a plurality of subunits, each subunit having, at least one reactive functionality; reacting the at least one reactive functionality with a functionalizing agent in a reaction media, the functionalizing agent binding to the at least one reactive group, and having at least one carboxylic acid moiety at a distal end of the functionalizing agent; and adjusting the pH of the reaction media wherein the at least one carboxylic acid moiety is protonated.

In a further aspect of the invention there is provided a pharmaceutical composition for administering a bioactive agent comprising; the bioactive agent in association with a pharmaceutically acceptable carrier, wherein the carrier is a biocompatible polymeric matrix for immobilizing a bioactive agent comprising: a biocompatible polymer comprising at least one polymer chain having a plurality of chain lengths and subunits in the chain lengths, each subunit having at least one reactive functionality and a plurality of pairs of first and second linkages, each pair of linkages extending between a pair of subunits on opposed spaced apart chain lengths, the first and the second linkages having a binding group covalently attached to the reactive functionality and at least one —COOH carboxylic acid moiety, distal the binding group, the at least one —COOH carboxylic acid group of a pair of first and second linkages being bondingly associated such that the chain lengths and the pairs of linkages define the matrix.

### BRIEF DESCRIPTION OF THE DRAWINGS

Further features and advantages of the present invention will become apparent from the following detailed description, taken in combination with the appended drawings, in which:

FIG. 1: X-ray diffractogram of native chitosan (NC), a chitosan succinate (CS), three chitosan succinic acid (CSA) at different degrees of substitution (DS) and at different pH, producing different levels of protonation of the carboxylic acid end groups;

FIG. 2: X-ray diffractogram of carboxymethylated chitosan at different pH's (4.5 and 7.0), with this derivative carried out under the same conditions as in Example 1 using monochloroaectic acid as functionalizing agent;

FIG. 3: Schematic presentation of the reaction between chitosan, and succinic anhydride, producing a functionalized chitosan polymeric matrix and a hypothetical presentation of the resulting molecules stabilization by dimers of carboxylic acid groups;

FIG. 4: FTIR spectra of native and succinylated chitosan;

FIG. 5: Release profiles of acetaminophen from tablets (500 mg) based on chitosan succinic acid with 15-20% degree of substitution, with 20 and 60% of drug loading;

FIG. 6: Release profiles of sodium benzoate from tablets (500 mg) based on chitosan succinic acid with degree of substitution higher than 95%, containing 20 and 60% of drug;

FIG. 7: Release profiles of Metformin from tablets (500 mg) based on chitosan succinic acid with degree of substitution higher than 95%, with 20% and 60% of drug loading;

FIG. 8: Release profiles of acetaminophen from tablets (500 mg) based on succinyl alginate with degree of substitution about 20% containing 20, 40 and 60% of drug loading; and

FIG. 9: Release profiles of acetaminophen from tablets (500 mg) based on succinyl ethylcellulose with degree of substitution 15-20%, with 20, 40 and 60% of drug loading.

### DETAILED DESCRIPTION OF THE PREFERRED EMBODIMENT

The present invention concerns a polymeric matrix which is biocompatible and/or biodegradable, and which can be used as a carrier and as a protective coating against digestion and denaturation of bioactive agents.

The matrix includes a non-toxic, biocompatible and/or biodegradable polymer. The polymer is in a preferred embodiment a natural or modified polysaccharide or oligosaccharide, that may include chitosan, alginate, pectin, agar, agarose, cellulose, cellulose derivatives and combinations thereof. In a preferred embodiment the polymer is chitosan. Chitosan is a linear polysaccharide composed of randomly distributed subunits of β-(1-4)-linked D-glucosamine (deacetylated unit) and N-acetyl-D-glucosamine (acetylated unit). The chitosan subunits have a reactive amine group.

The polymer is functionalized with the addition of linkages, by derivatization with groups having carboxylic acid (in a preferred range from pKa 3.0-5.0) to produce the polymeric matrix, or network, through a reaction with a functionalizing agent which produce linkages between polymer chains.

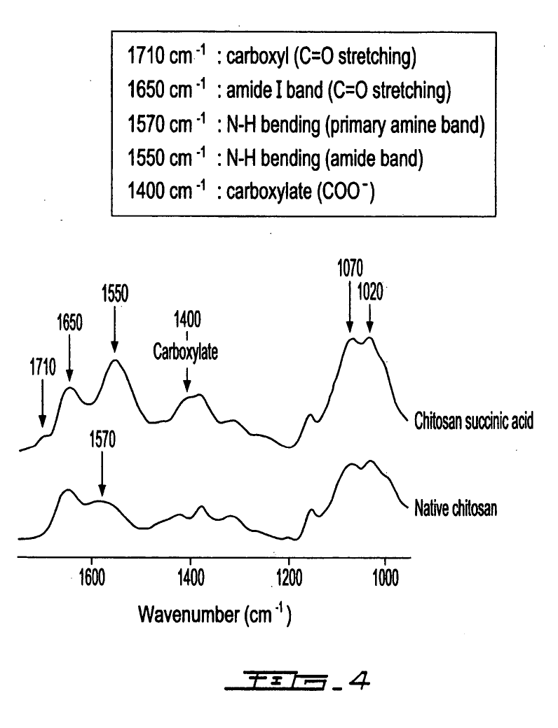

The polymeric matrix of the present invention may have one or more polymer chains, each chain having specific chain lengths. The chain lengths of the polymer chains interact and are believed to be found typically opposed to one another. These polymer chain lengths have one or more types of subunits to which the linkages are covalently attached via a reactive functionality. The matrix has the inherent property of being stabilized, it is believed that this stabilization is by a bonding association, typical understood as hydrogen bonding and other dipolar interactions. These interactions are believed to be via opposed carboxylic acids dimers, where the carboxylic acid is not ionized but in the form of a —COOH moiety (schematically represented in FIG. 3). These carboxylic acid dimmers link the polymer chain, and constitute a barrier limiting the access of gastric fluid thus protecting the sensitive bioactive agents during gastric transit (pH 1.2-2.0). It is also believed, that non-associated —COOH moieties (those that are not associated with another —COOH moiety) can associate with bioactive agents which find room the spaces or voids within the polymeric matrix between linkages of the —COOH moieties.

It has been found that the —COOH moieties, in their protonated form, produce excellent stabilization and immobilization of bioactive agents and protection from external denaturing factors. Furthermore the level of immobilization and protection is particularly high when compared with protection, stabilization and immobilization by the polymer without linkages. Here external factors are defined as those that occur primarily outside the polymeric matrix, and are those denaturing factors that are encountered for example in the stomachal passage.

Furthermore, the polymeric matrix with carboxylic functionalization is converted in an intestinal environment into a carboxylated soft hydrogel and releases the bioactive agents in a controlled way at action or absorption sites.

The present invention resides in the addition of functionalizing agents having or producing carboxylic acid moieties as side chains to polymers, where in a preferred embodiment these polymers are natural polymers. The functionalizing agents attach themselves to the polymer through reactive functionalities present in the subunits of the polymer. These reactive functionalities are generally amine or carboxylic groups which can be activated, although other reactive functionalities would be known to the skilled practitioner.

For this purpose, the carboxylation can be conducted with the following functionalizing agents: succinic anhydride; dimethylsuccinic anhydride dimethylglutaric anhydride; diacetylsuccinic anhydride; ethylenediaminetetraacetic dianhydride; diethylenetriaminepentaacetic (DETPA) dianhydride; monochloroacetic acid; phthalic anhydride and combinations thereof.

It is worth noting that the carboxyl groups of the present invention remain initially non-ionized, that is they are protonated or uncharged carboxylic acid groups, and not in the form of an ionized salt (a carboxylate), as previously described (Aiedeh, K. and Taha, M. O., 1999. Arch. Pharm. Pharm. Med. Chem., 332, 103-107; Bernkop-Schnurch, A. and Krajicek, M. E. 1998. J. Control. Release., 50, 215-223).

The uncharged carboxylic acid groups are considerably less hydrophilic compared to their charged counterparts (carboxylate anions) and protect the bioactive agent to which they are attached thus allowing for a longer release time for oral drug administration.

Furthermore, the carboxylic material dried under salt form requires a binding agent in their formulation to impart cohesive qualities. Aiedeh and Taha, (1999, Ibid.) reported a large quantity of lactose (more than 35% of the binding agent) added in the formulation of chitosan succinate based tablet. This addition of lactose is required so that the tables remain intact after compression. While on the other hand, the carboxylic acid polymeric matrix of the present invention have good cohesiveness and in a preferred embodiment little to no binding agent is needed in a formulation with a bioactive agent. In a preferred embodiment there would be 10% or less by weight of a binding agent, while in more preferred embodiment there would be 5% or less, while in the most preferred embodiment there is no binding agent.

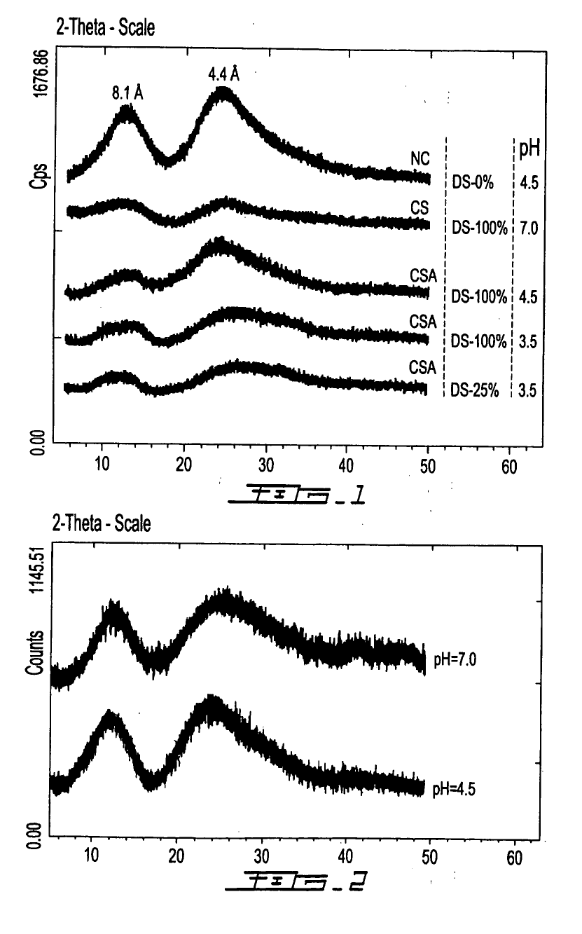

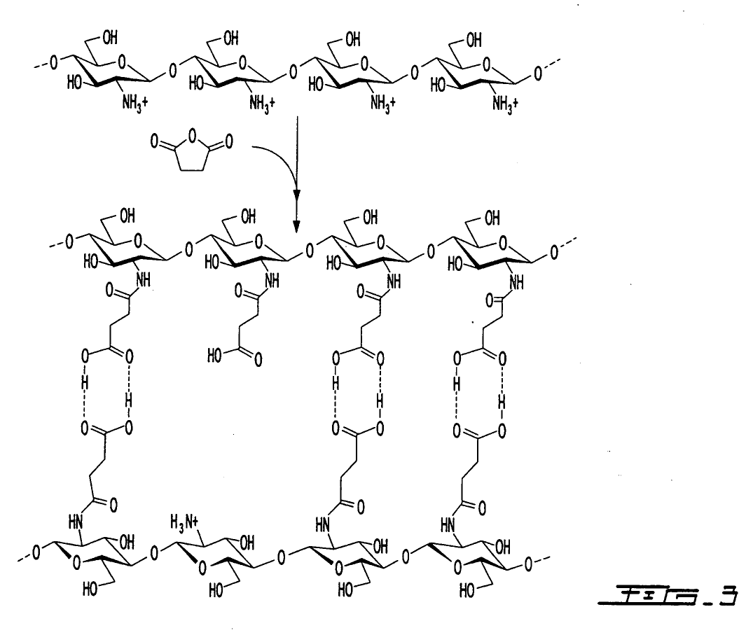

The structure of chitosan functionalized with carboxylic acid in protonated form, has greater crystallinity and a more stable structural organization than the carboxylate or salt form. This is illustrated in FIGS. 1 and 2.

The line upper most in the FIG. 1 shows the diffractogram pattern of native chitosan (NC, having no substituted groups or a degree of substitution-0%, DS-0%) at pH 4.5, having two peaks at 8.1 and 4.4 Å. These peaks of moderately high intensity and sharpness, predict native chitosan's insolubility in neutral medium (pH approx. 7.0).

The crystalline structure of chitosan was gradually altered with the presence of carboxyl groups. Four more X-ray diffractograms are presented in FIG. 1, which are variations of a the polymeric matrix of the present invention. In this case, a series of descending sequential substantially horizontal broad lines was observed. However, structure of the polymeric matrix a pH 4.5 has a more stable organization the other forms of chitosan succinic acid conjugate. The lines are identified (in descending order from the uppermost line representing native chitosan (NC)) as: a chitosane succinate (CS) matrix of DS-100% produced at a pH 7, where the carboxylic acid end groups are de-protonated; a chitosan succinic acid (CSA), with a DS-100% produced at pH 4.5, where roughly half the carboxylic acid end groups are not protonated (succinic acid has a pKa of 4.2); a CSA with a DS-100% produced at a pH of 3.5, where a majority of the carboxylic acid end groups are protonated; and the lowest line in FIG. 1 is a CSA, with only a degree of substitution of 25%, produced at pH 3.5, where, the carboxylic acids end groups are protonated but the non-substituted amine groups are also protonated to produce cations. The CS DS-100% at pH 7.0 line shown shows peaks of moderately low intensity and that are broader than those of chitosan succinic acid (pH 4.5), this indicates that the CSA of 4.5 is more crystalline and has a more stable organization.

FIG. 2 shows that a similar phenomenon was observed for carboxymethylated chitosan. The likely explanation for these differences between the protonated carboxylic acid and the ionized carboxylate, is based on the formation of a network enhanced stabilization, which may be due to the dimer of carboxylic acid residues of neighboring chains and hydrogen bonding (dipole-dipole attraction as represented in FIG. 3) as well as, other dipolar interactions. This stabilized network likely hinders the hydration of gastric acid solution or the access of denaturing factors through the polymeric matrix. In the intestinal environment, the carboxylic residues are gradually deprotonated giving an adhesive gel character providing an intimate contact with the absorbing membrane and a prolonged residence time in the intestine.

These carboxylic polymer matrices can be used as an excipient or as a coating, which are compact, mechanically stable and in acidic environment able to protect the bioactive agents during gastric transit through the stomach. After passage through the stomach the matrices are able to release bioactive agent selectively in the intestinal tract, at the required site of action.

The polymeric matrix of the present invention can be further processed to a dry powder for preparation of tablets (the preferred form in pharmaceutics industries due to their simplicity and low cost). Moreover, the administration <<per os>> (oral administration), is considered as the most natural, simplest and safest way of administering bioactive agents. Tablet manufacturing by direct compression consists of mechanically mixing of a drug powder with the polymeric matrix and/or an excipient powder and then compressing the mixture under suitable pressure, this process would be understood by the skilled practitioner.

The release mechanism of the bioactive agent is likely based on diffusion, swelling or erosion, followed by the dissolution of the active compounds.

The polymeric matrices of the present invention can be put into different formulations depending on the intended application and route of administration. The formulations suggested in the present invention may be used in various delivery systems including beads, microbeads, tablets, capsules, etc. for oral dosage. While nanoparticles may be produced for parenteral administration, implants for subcutaneous devices and film, creams and emulsions for topical administration and films and coatings for food protection.

Thus, in a preferred embodiment a modified polysaccharide (such as chitosan, alginate and cellulose and its derivatives) functionalized with carboxylic groups to produce the polymeric matrix type of the present invention is formulated with a bioactive agent, to protect the bioactive agent from denaturing factors of the external harmful environment, as well as to control the site and the rate of its release. Particularly in the case of bioactive agents as peptides or proteins for delivery in gastrointestinal tract, soybean proteins can be added in the formulation in order to reduce the proteases activities due to the presence of trypsin inhibitor (Kunitz, M. 1946. J. Gen. Physiol., 30, 311-320; Kunitz, M. 1946. J. Gen. Physiol., 30, 291-310). In addition, the soybean proteins could also serve as a competitive substrate and reduce the attack of proteases on the bioactive peptides or proteins.

The bioactive agents can be defined as agents having an effect on a biological system. The bioactive agent may be a drug, an alkaloid, a DNA, an RNA, a hormone, a nutraceutic product, a vitamin, a mineral, a probiotic, a bacterium, cells, a bacteriocine, an enzyme, a bioactive peptide, a protein, an antioxidant, an antimicrobial, an antifungal, an antiparasitic agent, a pesticide and combinations thereof.

Additionally, the functionalized polymers are able to form bactericide free-standing films. Consequently, there is great interest to using them as coating or packaging films for food protection and preservation. Moreover, the films prepared from carboxyl chitosan also have good mechanical properties allowing their use as a transdermal patch or adhesive membrane for the mucosa.

For agriculture applications, these modified polymers matrices can be used as support to entrap pesticides. The polymeric matrices have several advantages: they are generally natural or derived from natural sources, they are non-toxic, biocompatible, and usually biodegradable matrices; they lower and control the release rate of bioactive agents; they may stimulate activity of plants due to chitosan (matrix) which is able to improve disease resistance and to immunize plants to kill many fungi, bacteria and viruses (Rabea, E. I. et al., 2003. Biomacromolecules, 4, 1457-1465; Doares, S. H. et al., 1995. Proc. Natl. Acad. Sci. USA., 92, 4095-4098).

The chitosan, alginate or cellulose are used in preferred embodiment of the present invention, as the precursor for the polymeric matrices. The chitosan may be obtained from chitin after deacetylation, and is mainly composed of 2-amino-2-deoxy-β-D-glucopyranose repeating units but still retaining a small amount of 2-acetamido-2-deoxy-β-D-glucopyranose residues. Chitosan amino groups (at C2 position) are nucleophilic and reactive, suitable for chemical modifications (Monteiro Jr, O. A. C. and Airoldi, C. 1999. Int. J. Biol Macromol., 26, 119-128).

The alginate is a natural polysaccharide extracted from seaweed. It is a copolymer with alternating sequences of β-D-mannuronic and α-L-guluronic acid residues, 1,4-glycosidically linked. The alginate can be modified by substitution of hydroxyl groups with succinic anhydride, ethylendiamintetraacetic dianhydride or monochloracetic acid, etc. It is worth mentioning that other polysaccharides such as agarose, carrageenan, hyaluronane, cellulose and combination thereof and derivatives, etc. can be modified as described for alginate.

### EXAMPLE 1

### Monolithic Drug Controlled Release Dosage Forms Based on Chitosan Succinic Acid Conjugate

Chitosan succinic acid was synthesized at pH 4.5 and at temperature 60° C. for a minimum of 3 h. A quantity of 20.0 g chitosan was dissolved in 900 mL of water containing 11.5 mL of lactic acid (or acetic acid). When the chitosan is completely homogenized, an amount in the range of 0.5 -20 g of succinic anhydride (dissolved in methanol, ethanol or acetone just prior to use) is slowly added. After the reaction, the final pH of solution is adjusted to 4.5 and the volume is completed to 1 L with distilled water. Similarly, chitosan succinic acid can be obtained by varying the conditions of starting chitosan whiting (1-20 g chitosan dissolved in 900 mL of organic acid) or whiting pH of the medium (pH 3.5-6.5).

To obtain the powder, the solution was precipitated in methanol or ethanol and dried 3-4 times in acetone. Alternatively, the powders can be obtained by using a spray-drying process. This process presents several advantages: speed and low cost; No solvent is used; for certain hydrosoluble drugs (i.e. acetaminophen) can be added before the drying process in the polymeric matrix solution to obtain a homogeneous dry powder mixture.

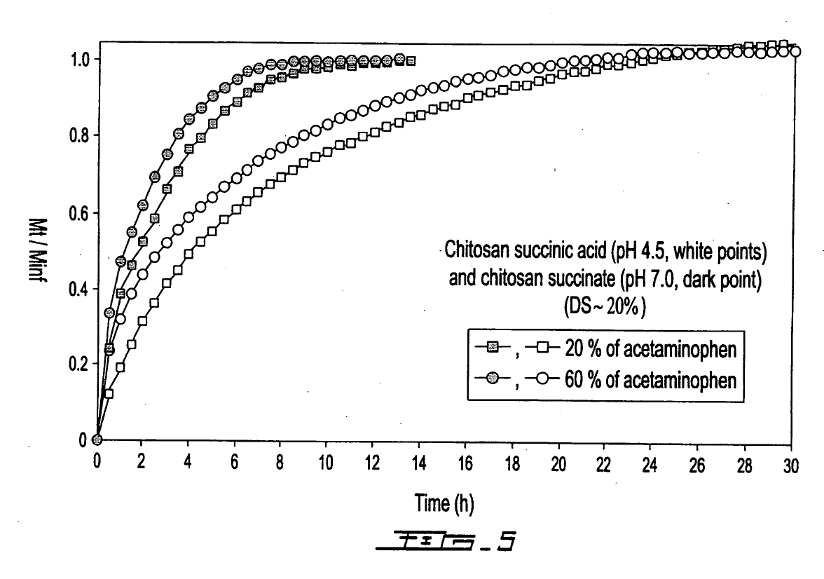

The degree of substitution (DS) is the molar percentage of functionalizing agent attached to the reactive functionalities divided by the total amount of reactive functionalities available. The degree of substitution, DS, for chitosan may be determined by the colorimetric method (ninhydrine assay) as described by Curotto and Aros (Curotto, E. and Aros, F. 1993. Anal. Biochem., 211, 240-241) and by the titration method as described by Le-Tien et al. (2003) Ibid.). From Fourier transform infrared (FTIR) analysis (FIG. 4), the absorption peaks at 1650 cm⁻¹ can be assigned to the carbonyl stretching of secondary amides (amide I band), at 1570 cm⁻¹ to the N—H bending vibration of 2-aminoglucose primary amines, and at 1550 cm⁻¹ to the N—H bending vibrations (amide II band). The native chitosan (NC), nonmodified is used as a starting material and is reported by the manufacturer to have a degree of deacetylation of 85-89%. The absorption bands at 1650, 1570, and 1550 cm⁻¹ confirm the presence of both 2-aminoglucose and 2-acetamidoglucose repeat units. After N-succinylation, the vibrational band corresponding to primary amino groups at 1570 cm⁻¹ disappeared, while prominent bands at 1650 and 1550 cm⁻¹ were observed. In addition, the appearance of band at 1710 cm⁻¹ is attributed to the carbonyl group and the increase of bands at 2850 -2950 cm⁻¹, is ascribed to —CH₂— groups of succinyl chains.

Monolithic tablets (500 mg, 12.5 mm diameter, 3.0 mm thickness) of native or succinylated chitosan containing 20, 40, 60 and 80% of drugs were produced by direct compression of powders (2.3 T/cm² in a Carver hydraulic press). The drugs used as tracers were acetaminophen (neutral polar drug), Metformin (positively charged drug) and sodium benzoate (negatively charged drug). Tablets with increasing drug loading (20, 40, 60 and 80%) were prepared for chitosan succinic acid with a DS (degrees of substitution) of approximately about 20, 50 and 80%. The kinetics of drug release were recorded using a Distek dissolution 2100A paddle system (50 rpm) coupled with an UV Hewlett Packard spectrophotometer for detection of acetaminophen and sodium benzoate (280 nm) or Metformin (250 nm) and presented using the diffusion equation as the ratio of the amount of drug released at the time t (Mt) and the total amount (M.inf, amount of drug released at time ∞) of drug released from the tablet. The dissolution medium was 1 L of 50 mM phosphate buffer (pH 7.2) at 37° C.

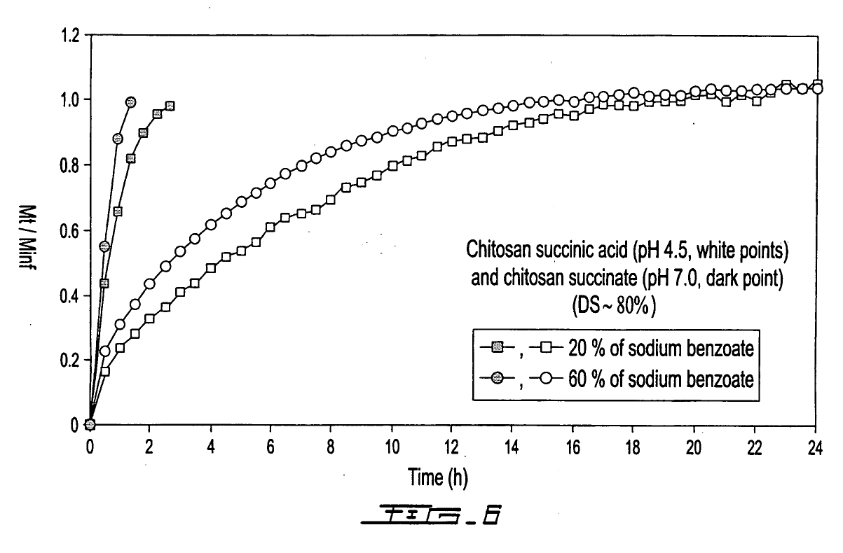

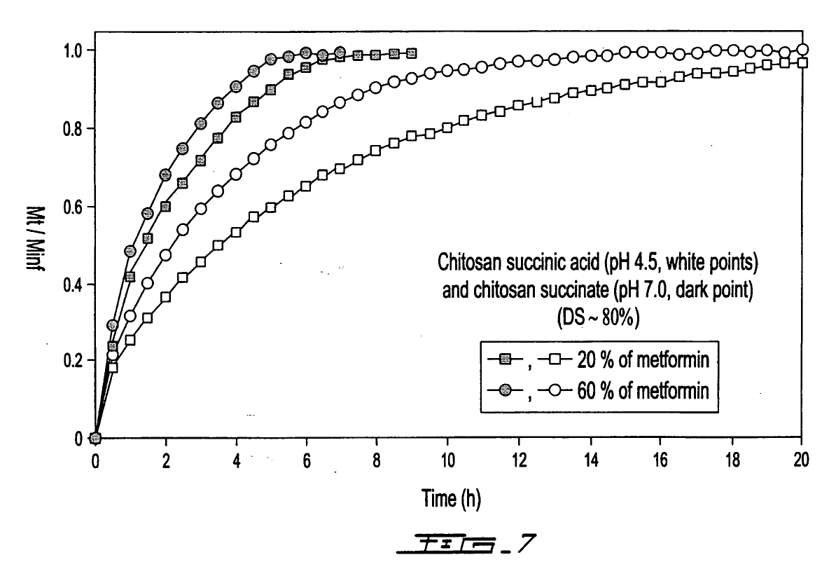

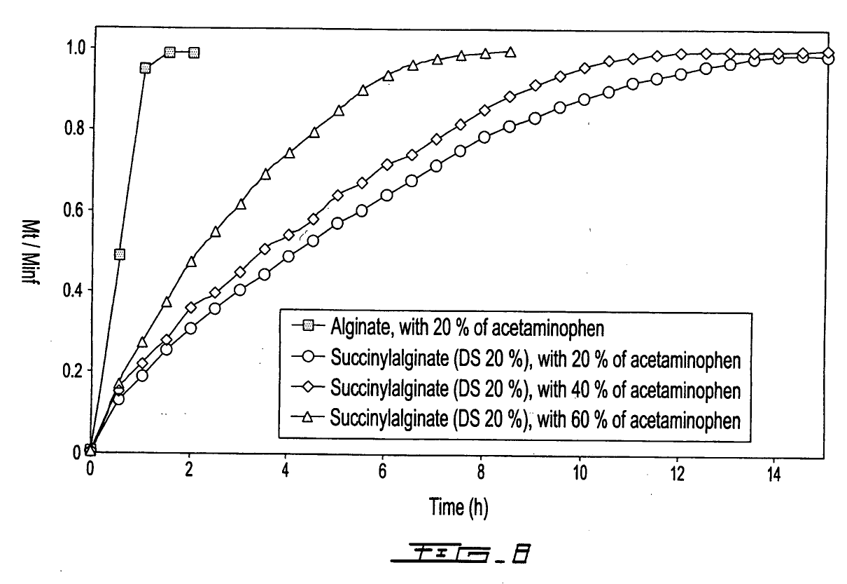

For the native chitosan, the tablets are rapidly disintegrated and their contents released within 1 h, whereas the released times of tablets including the chitosan succinate polymeric matrix were between 4-7 hours, while chitosan succinic acid-based tablets remained intact and a long release, greater than 12 hours, was observed (FIG. 5, 6 and 7). For neutral and polar drugs (acetaminophen), best results were observed for tablets of chitosan succinic acid with degree of substitution about 15-20% (release over 20 h). In contrast, for charged drugs (sodium benzoate and Metformin), chitosan succinic acid tablets with degree of substitution superior to 80% showed longer (release time 12-14 h) than tablets with low degree of substitution.

### EXAMPLE 2

### Succinylalginate Used as Matrix for Drug Controlled Release

The succinylalginate was synthesized at pH 8.0-10.0 and at temperature 60° C. by treating alginate with succinic anhydride for a minimum of 3 h. Indeed, a quantity of 20.0 g of alginate sodium salt was dissolved in 900 mL of distilled water containing 1.5% of NaOH. When the solution is completely homogenized, an amount of 0.5-20 g of succinic anhydride (dissolved in methanol, ethanol or acetone just prior to use) is slowly added. After the reaction, the final pH of solution is adjusted to 4.5 with lactic acid or HCl (0.1-1.0M) and the final volume is completed to 1 L with distilled water. To obtain the powder, the solution can be precipitated and dried in acetone or by spray drying.

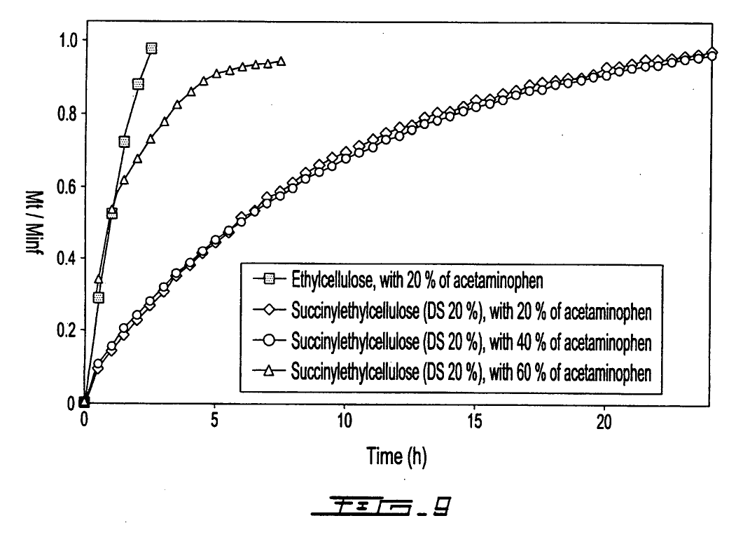

With the native alginate (non-modified) as excipient, the tablets are easily swollen, adhesive and released of the acetaminophen within 2-3 h, whereas the succinylalginate (DS˜20%) tablets are degraded gradually and a long release time was observed (FIG. 8). Best results were obtained with 20 and 40% of drug loading which gave a release time of 12 hours.

### EXAMPLE 3

### Succinylethylcellulose as Matrix for Drug Controlled Release

The succinylethylcellulose was synthesized as described to succinyl alginate (Example 2). With the native hydroxyethylcellulose (HEC, non-modified), the tablets were rapidly disintegrated and the acetaminophen released within 1 h, whereas the succinylethylcellulose (DS˜20%) tablets are gradually degraded and a long release time (more than 20 h) was observed for matrices with 20 and 40% of drug loading (FIG. 9).

### EXAMPLE 4

### Chitosan Ethylenediaminetetraacetic (EDTA) Acid Conjugate as Matrix for Drug Controlled Release

The chitosan EDTA acid was synthesized as described in Example 1 to obtain the powder with the DS 15-95%. An amount of 20 g of chitosan was treated with an amount in a range of 0.2-10 g of EDTA dianhydride. Similar properties as those obtained with chitosan succinic acid excipients were observed. The release time of acetaminophen from the EDTA polymeric matrix having a degree of substitution of about 15%, was 18 hours, whereas the release times for Metformin or sodium benzoate were between 10-12 hours. These tablets including a EDTA polymeric matrix of the present invention were found to have, an adhesive behaviour more significant than chitosan succinic acid polymeric matrix based tablets.

### EXAMPLE 5

### Chitosan Succinic Acid Based-tablets for Probiotic Protection

In order to evaluate the efficiency of the polymeric matrix in the gastrointestinal system, Lactobacillus or other probiotics may be formulated with our modified chitosan polymeric matrix. In this example, Lactobacillus rhamnosus is used due to its sensitivity at pH<3.0.

Matrix synthesis—A matrix of chitosan succinic acid (DS approx. 20-80%) was synthesized as described in Example 1, with the final pH of solution adjusted to 5.5. Tablets of chitosan succinic acid containing approximately 10⁸ CFU (Colony Forming Units)/mg of lactic bacteria powders were obtained by direct compression (2.3 T/cm²).

Stability of entrapped bacteria in Simulated Gastric Fluid (SGF)—Tablets (containing lactic bacteria) or free lactic bacteria powders (without excipient) were incubated at 37° C. in simulated gastric fluid (the SGF was obtained according to United States Pharmacopeia). The SGF contained 3.2 g/L of pepsin (approx. 600 units/mg) and 2.0 g/L of NaCl and pH finally adjusted to 1.5 using HCl (1.0 M) solution. SGF was sterilized by filtration with a Bottle Top Vacuum Filter (0.2 μm pore size; Nalge Nunc International, New York, N.Y., U.S.A.). After 1-2 hours in the simulated gastric fluid, tablets were collected and transferred in 100 mL of sterile phosphate buffer (0.5 M, pH 7.5) with mild shaking (100 rev./min) in a G24 Environmental Incubator Shaker (New Brunswick Scientific Co., New Brunswick, N.J., U.S.A). For free bacterial cells in SGF, a volume of 1.0 mL of bacterial suspension was added in the same phosphate buffer. A similar procedure was applied to tablets or free cells, without SGF treatment, followed by the determination of initial bacterial number.

Determination of viable cells—Appropriate dilutions from these samples (free cells and tablets dissolved in phosphate buffer solution, with or without treatment in SGF) were conducted in sterile peptone water (0.1%, w/v) and poured onto Lactobacilli MRS agar plates. Plates were incubated aerobically at 37° C. for 48 h. The average number of CFU was determined by Darkfield Quebec Colony Counter 3330 (American Optical Company, New York, N.Y., U.S.A.).

The preliminary results with L. rhamnosus show a surviving fraction of about 70% when formulated with the matrix of the present invention, whereas the viability of the free bacteria was 1%, after 1 hour of incubation in the gastric phase (pH 1.5) at 37° C.

### EXAMPLE 6

### Chitosan Succinic Acid Used as a Matrix to Immobilize Bioactive Molecules in Microbeads (Oral Administration) or Nanoparticles (Parenteral Administration)

The immobilization of a bioactive molecule into spheres can be achieved by first producing an emulsification followed by gelation. Indeed, the mixture (bioactive agents/functionalized chitosan polymeric matrix) was introduced into the oil phase (i.e. Canola oil) with moderate stirring to create an emulsion (Le-Tien et al., 2004. Biotechnol. Appl. Biochem., 39, 347-354). These emulsions were then gelled by the addition of salt of organic acids (i.e. sodium lactate, sodium citrate or sodium acetate, etc.) and the microbeads obtained were separated by simple centrifugation at low speed. The oil can be extracted with solvents and eliminated by successive washings in distilled water. It is also of interest to note that the degree of substitution can play a significant role in the mechanical properties of microbeads. The microbead forming solution was prepared at concentrations of 1.0-3.0% of chitosan succinic acid.

To obtain the nanoparticle, the same process was used, but with the cotton oil or others having the same viscosity.

To evaluate the matrices, the catalyst was immobilized in microbeads and the retained activity was determined after 30 min of incubation in simulated gastric fluid with pepsin (pH 1.5). The catalytic activity was spectrophotometrically determined at 240 nm by monitoring the decrease of absorbance caused by the decomposition of hydrogen peroxide during catalysis (Aebi, H. E. 1987. In: Method of enzymatic analysis. Bermeyer, H. U., Bermeyer, J. and Grabl. M. Editors, Vol. III, VCH Publisher, New York, 273-277; Le Tien et al., 2004. Biotechnol. Appl. Biochem., 39, 189-198).

The results showed that the retained activities (after 30 min in acid, pH 1.5) for free catalase was 2.5%, whereas those of catalase entrapped in microbeads and nanoparticles were 55 and 32% respectively. These results suggest that the matrix has a protective effect against acidity and pepsin denaturation.

### EXAMPLE 7

### Use of the Chitosan Succinic Acid as a Matrix for Transdermal Bioactive Agent Delivery

The functionalized chitosan polymeric matrix was synthesized as previously described with a degree of substitution of about 60-80%. 500 mg of caffeine was added in 100 mL functionalized chitosan polymeric matrix solution (2.5%) and stirred during 30 min at 80° C. for 2 h. Films were produce by applying 20 mL of the solution evenly onto Petri dishes and dried at room temperature for 24-48 h.

The film produced was subjected to an in vitro diffusion test using Franz apparatus, with an exposed surface area of 3.9 cm². Polydimethylsiloxan (Silastic®) membranes have been used as barrier and the receptor compartment (dissolution phase) was 1 L of 50 mM phosphate buffer (pH 7.2) at 32° C. At predetermined time intervals, the samples (5 mL aliquots) were analyzed at 272 nm for the determination of caffeine permeation.

The results show that there is a penetration of the caffeine, and amount of 405 μg/cm² of caffeine was measured in the receptor compartment after 24 h.

The embodiment(s) of the invention described above is(are) intended to be exemplary only. The scope of the invention is therefore intended to be limited solely by the scope of the appended claims.

1. A biocompatible polymeric matrix for immobilizing a bioactive agent comprising:
a biocompatible polymer comprising at least one polymer chain having
a plurality of chain lengths and subunits in the chain lengths,
each subunit having at least one reactive functionality and
a plurality of pairs of first and second linkages, each pair of linkages extending between a pair of subunits on opposed spaced apart chain lengths,
the first and the second linkages having a binding group covalently attached to the reactive functionality and
at least one —COOH carboxylic acid moiety, distal the binding group,
the at least one —COOH carboxylic acid group of a pair of first and second linkages being bondingly associated such that the chain lengths and the pairs of linkages define the matrix.

2. The matrix of claim 1, wherein the biocompatible polymer is a natural or modified polysaccharide or oligosaccharide.

3. The matrix of claim 2, wherein the natural or modified polysaccharide or oligosaccharide, is selected from the group consisting of chitosan, alginate, pectin, agar, agarose, cellulose, cellulose derivatives and combinations thereof.

4. The matrix of claim 3, wherein the natural or modified polysaccharide or oligosaccharide, is chitosan, alginate or combinations thereof.

5. The matrix of claim 1, wherein the functionalizing agent and the at least one reactive functionality are attached in accordance with a degree of substitution.

6. The matrix of claim 5, wherein the degree of substitution is varies between 1 and 100%,
wherein the degree of substitution is the molar percentage of functionalizing agent attached to reactive functionalities divided by the total amount of the reactive functionalities.

7. The matrix of claim 1, wherein the functionalizing agent is selected from the group consisting of succinic anhydride; dimethylsuccinic anhydride dimethylglutaric anhydride; diacetylsuccinic anhydride; ethylenediaminetetraacetic dianhydride; diethylenetriaminepentaacetic (DETPA) dianhydride; monochloroacetic acid; phthalic anhydride and combinations thereof.

8. The matrix of claim 1, comprising an immobilized bioactive agent.

9. The matrix of claim 8, wherein the bioactive agent is selected from the group consisting of a drug, an alkaloid, a DNA, an RNA, a hormone, a nutraceutic product, a vitamin, a mineral, a probiotic, a bacterium, cells, a bacteriocine, an enzyme, a bioactive peptide, a protein, an antioxidant, an antimicrobial, an antifungal, an antiparasitic agent, a pesticide and combinations thereof.

10. The matrix of claim 8, wherein the bioactive agent is selected from the group consisting of acetaminophen, Metformin, and sodium benzoate.

11. A method for preparing a biocompatible polymeric matrix comprising:
providing a biocompatible polymer comprising a plurality of subunits, each subunit having,
at least one reactive functionality;
reacting the at least one reactive functionality with a functionalizing agent in a reaction media, the functionalizing agent binding to the at least one reactive group, and having at least one carboxylic acid moiety at a distal end of the functionalizing agent; and
adjusting the pH of the reaction media wherein the at least one carboxylic acid moiety is protonated.

12. The method of claim 11, wherein the matrix is precipitated and dried from the reaction media to produce a powder.

13. The method of claim 11, wherein matrix is obtained from the reaction media via spray-drying to produce a powder.

14. The method of claim 11, wherein the biocompatible polymer is a natural or modified polysaccharide or oligosaccharide.

15. The method of claim 14, wherein the natural or modified polysaccharide or oligosaccharide, are selected from the group consisting of chitosan, alginate, pectin, agar, agarose, cellulose, cellulose derivatives and combinations thereof.

16. The method of claim 15, wherein the natural or modified polysaccharide or oligosaccharide, is chitosan, alginate or combinations thereof.

17. The method of claim 11, wherein the functionalizing agent and the at least one reactive functionality are attached in accordance with a degree of substitution.

18. The method of claim 17, wherein the degree of substitution is varies between 1 and 100%,
wherein the degree of substitution is the molar percentage of functionalizing agent attached to reactive functionalities divided by the total amount of the reactive functionalities.

19. The method of claim 11, wherein the functionalizing agent is selected from the group consisting of succinic anhydride; dimethylsuccinic anhydride dimethylglutaric anhydride; diacetylsuccinic anhydride; ethylenediaminetetraacetic dianhydride; diethylenetriaminepentaacetic (DETPA) dianhydride; monochloroacetic acid; phthalic anhydride and combinations thereof.

20. A pharmaceutical composition for administering a bioactive agent comprising;
the bioactive agent in association with a pharmaceutically acceptable carrier, wherein the carrier is a biocompatible polymeric matrix for immobilizing a bioactive agent comprising:
a biocompatible polymer comprising at least one polymer chain having
a plurality of chain lengths and subunits in the chain lengths,
each subunit having
at least one reactive functionality and
a plurality of pairs of first and second linkages, each pair of linkages extending between a pair of subunits on opposed spaced apart chain lengths,
the first and the second linkages having a binding group covalently attached to the reactive functionality and
at least one —COOH carboxylic acid moiety, distal the binding group,
the at least one —COOH carboxylic acid group of a pair of first and second linkages being bondingly associated such that the chain lengths and the pairs of linkages define the matrix.

21. The pharmaceutical composition of claim 20, wherein the bioactive agent is selected from the group consisting of acetaminophen, Metformin, and sodium benzoate.

22. The pharmaceutical composition of claim 20, wherein the bioactive agent is a drug, an alkaloid, a DNA, an RNA, a hormone, a nutraceutic product, a vitamin, a mineral, a probiotic, a bacterium, cells, a bacteriocine, an enzyme, a bioactive peptide, a protein, an antioxidant, an antimicrobial, an antifungal, an antiparasitic agent, a pesticide and combinations thereof.

23. The pharmaceutical composition of claim 20, in a tablet form, a sphere form, a films, in a hydrogel or in an emulsion.
